(19) United States
(12) Patent Application Publication
(10) Pub. No.: US 2025/0186680 A1
(43) Pub. Date: Jun. 12, 2025

(54) DEVICE FOR EAR AND RELATED METHODS

(71) Applicant: QUENTIA LLC, San Diego, CA (US)

(72) Inventors: Elizabeth Nation, San Diego, CA (US); Javan Nation, San Diego, CA (US); Colin Zyskowski, Spring Valley, CA (US); Mauricio de Oliveira, San Diego, CA (US)

(21) Appl. No.: 19/054,583

(22) Filed: Feb. 14, 2025

Related U.S. Application Data
(63) Continuation of application No. PCT/US2024/040069, filed on Jul. 29, 2024.
(60) Provisional application No. 63/529,604, filed on Jul. 28, 2023.

(51) Int. Cl. A61M 3/02; A61B 1/227

(52) U.S. Cl. CPC A61M 3/0283; A61B 1/227; A61M 3/0258; A61M 3/0262; A61M 2206/16; A61M 2210/0662

(57) ABSTRACT

This disclosure provides a device can be used to safely apply media to an ear. The device includes an insertion portion that is to be inserted into an ear canal. The insertion portion includes an outer wall to face the ear canal when the insertion portion is inserted into the ear canal, a supply channel to supply media to the ear canal when the insertion portion is inserted into the ear canal, and a drain channel to drain the media supplied to the ear canal. The supply channel is disposed to be closer to the outer wall than the drain channel.

### CROSS-REFERENCE TO RELATED APPLICATION

This application is a continuation of International Application No. PCT/US2024/040069, filed Jul. 29, 2024, which claims benefit of and priority to U.S. Provisional Application No. 63/529,604, filed on Jul. 28, 2023, the entire disclosure of which is hereby incorporated by reference in its entirety.

### FIELD

This disclosure relates to devices and methods for an ear, and particularly for devices configured to apply media to an ear.

### BACKGROUND

Earwax, also known as cerumen, is a natural substance produced by specialized glands in the skin of the ear canal to help protect from dirt, debris, and infection. For the majority of people, earwax is naturally expressed out of the ear canal and does not need removal. In some cases, however, cerumen can build up in the ear canal causing a cerumen occlusion and cause symptoms such as hearing loss, ear pain, ear fullness, and the inability for healthcare providers to see the tympanic membrane (ear drum) through the ear canal. Hearing aid users are an example of a patient population that are at higher risk of cerumen occlusion because the placement of hearing aids pushes and impacts cerumen deep into the ear canal. Young children are at higher risk of recurrent ear infections, and because of their small ear canals, even a small amount of cerumen can prevent a healthcare provider's ability to see the tympanic membrane and correctly diagnose a middle ear infection. For these mentioned populations and others, cerumen needs to be removed from the ear canal, which can be a difficult task for individuals as well as healthcare providers.

### SUMMARY

This Summary is provided to introduce a novel device that flushes cerumen out of the ear canal in an effective and non-painful way. Details are further described below in the Detailed Description. This Summary is not intended to identify key aspects or essential aspects of the claimed subject matter.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

The present disclosure is related to a device, wherein the device may comprise a body having an insertion portion to be inserted into an ear canal, wherein the insertion portion may include an outer wall to face the ear canal when the insertion portion is inserted into the ear canal, a supply channel to supply media to the ear canal when the insertion portion is inserted into the ear canal, and a drain channel to drain the media supplied to the ear canal, wherein the supply channel may be disposed to be closer to the outer wall than the drain channel.

The present disclosure is related to a device, wherein the device may comprise a body having an insertion portion to be inserted into an ear canal, wherein the insertion portion may include an outer wall to face the ear canal when the insertion portion is inserted into the ear canal, an inner wall forming a space in between the outer wall and the inner wall, a supply channel formed in the space in between the outer wall and the inner wall, the supply channel to supply media to the ear canal when the insertion portion is inserted into the ear canal, and a drain channel formed on the inner side of the insertion portion, the drain portion to drain the media supplied to the ear canal.

The present disclosure is related to a device, wherein the device may comprise a body having an insertion portion to be inserted into an ear canal, wherein the insertion portion may include an outer wall to face the ear canal when the insertion portion is inserted into the ear canal, an inner wall facing an inner side of the insertion portion, a supply channel formed in between the outer wall and the inner wall, the supply channel to supply media to the ear canal when the insertion portion is inserted into the ear canal, and a drain channel formed on the inner side of the insertion portion, the drain channel to drain the media supplied to the ear canal.

In some embodiments, the supply channel may include a portion to direct a flow of the media from the supply channel to generate shear stress on a surface of the ear canal.

In some embodiments, the supply channel may include a portion to direct a flow of the media from the supply channel to be angled toward a surface of the ear canal.

In some embodiments, the supply channel may include centrifugal canals to direct a flow of the media from the supply channel to be in a swirling motion.

In some embodiments, the supply channel may comprise a plurality of supply channels, and wherein the plurality of supply channels may be disposed to be around the circumference of the drain channel.

In some embodiments, the device may further comprise a media container coupled to the body, the media container to contain the media to be supplied to the supply channel.

In some embodiments, the media container may be squeezable to supply the media to the supply channel.

In some embodiments, the media container may be a squeezable bulb to be squeezable to supply the media to the supply channel.

In some embodiments, the device may further comprise a pump to apply a pressure to the media container to supply the media to the supply channel.

In some embodiments, the pump may be an electric pump.

In some embodiments, the pump may include a pedal to be pressable to generate the pressure.

In some embodiments, the device may further comprise a coupler to couple the device to a secondary device.

In some embodiments, the secondary device may be an otoscope.

### BRIEF DESCRIPTION OF THE DRAWINGS

In this Application:

FIG. 1 depicts a device body having an insertion portion to be inserted into an ear canal according to an embodiment of the disclosure.

FIG. 2 depicts a blown-up sectional portion as shown at numeral 2 of FIG. 1.

FIG. 3 depicts an alternate view of the device body of FIG. 1.

FIG. 4 depicts a cross-sectional view of the device body according to an embodiment of the disclosure. The left portion of FIG. 4 shows the device body; the right portion of FIG. 4 shows a blown-up portion from the corresponding left portion.

FIG. 5 depicts a cross-sectional view of the device body according to an embodiment of the disclosure.

FIG. 6 depicts a cross-sectional view of the device body according to an embodiment of the disclosure.

FIG. 7 depicts a cross-sectional view of the device body according to an embodiment of the disclosure.

FIG. 8A depicts an end view of the device body according to an embodiment of the disclosure.

FIG. 8B depicts an end view of the device body according to an embodiment of the disclosure.

FIG. 9 depicts an embodiment of the disclosure whereby a device body is constructed by coupling an inner component and an outer component.

FIG. 10 depicts an embodiment of the disclosure whereby the device body is coupled to a media container.

FIG. 11 depicts an embodiment of the disclosure whereby the device body is coupled to a media container.

FIG. 12 depicts an embodiment of the disclosure whereby the device body is coupled to a media container.

FIG. 13 depicts an embodiment of the disclosure whereby the device body is coupled to a media container.

FIG. 14 depicts an embodiment of the disclosure whereby the device body is coupled to a media container.

FIG. 15 depicts an end view of the media container where the device body attaches to the bulb.

FIG. 16 depicts a pump that can be used in in conjunction with the device body according to an embodiment of the disclosure.

FIG. 17 depicts a pump that can be used in in conjunction with the device body according to an embodiment of the disclosure.

FIG. 18 depicts a foot pedal that operate a pump according to an embodiment of the disclosure.

FIG. 19 depicts a foot pedal that operate a pump according to an embodiment of the disclosure.

FIG. 20 depicts an otoscope coupled to the device body according to an embodiment of the disclosure.

### DETAILED DESCRIPTION

A detailed description of one or more embodiments of the invention is provided below along with accompanying figures that illustrate the principles of aspects of the inventions disclosed herein. The inventions are described in connection with certain embodiments, but the invention or inventions are not limited to any particular embodiment. The scope of invention is limited only by the claims. Numerous specific details are set forth in the following description in order to provide a thorough understanding of aspects of the inventions. These details are provided for the purpose of non-limiting examples and the invention or inventions may be practiced according to the claims without some or all of these specific details.

### Overview of Disclosure

Earwax, also known as cerumen, is a natural substance produced by specialized glands in the skin of the ear canal to help protect from dirt, debris, and infection. For the majority of people, earwax is naturally expressed out of the ear canal and does not need removal. In some cases, cerumen can build up in the ear canal causing a cerumen occlusion and cause symptoms such as hearing loss, ear pain, ear fullness, and the inability for healthcare providers to see the tympanic membrane (ear drum) through the ear canal. Hearing aid users are an example of a patient population that are at higher risk of cerumen occlusion because the placement of hearing aids pushes and impacts cerumen deep into the ear canal. Young children are at higher risk of recurrent ear infections, and because of their small ear canals, even a small amount of cerumen can prevent a healthcare provider's ability to see the tympanic membrane and correctly diagnose a middle ear infection. For these mentioned populations and others, cerumen needs to be removed from the ear canal, which can be a difficult task for individuals as well as healthcare providers.

The present disclosure is also directed to treating different ear conditions that can affect people of all ages. Some of the most common ear conditions include ear infections, tinnitus, foreign organisms in an ear canal such as an insect, hearing loss, Meniere's disease, Barotrauma, etc.

Ear wax build up in the ears can cause discomfort and loss of hearing. The device or devices disclosed herein will allow an individual or physician to remove the cerumen in a comfortable and safe way. The market is saturated with ineffective, uncomfortable and sometimes even dangerous ear wax removal options. The device or devices disclosed herein provide an effective, comfortable and safe alternative for all providers.

In some embodiments, devices for an ear care, e.g., for delivering a drug, a medicinal effect, or a cleaning effect can be provided.

In some embodiments, the device can irrigate an ear to treat conditions. For example, the device with a pressurized water flow cab can irrigate the ear and cause built up wax to come loose and flush out.

Existing ear irrigation devices deliver a powerful stream of water directly at the eardrum, which can be uncomfortable and even painful. Especially in the pediatric world, children cannot handle the related discomfort.

### Definitions and Interpretation

Unless otherwise defined, all technical and scientific terms used herein have the same meaning as commonly understood by a person of ordinary skill in the art to which the present invention or inventions pertains. As used herein, and unless stated otherwise or required otherwise by context, each of the following terms shall have the definition set forth herein.

Other examples of implementations will become apparent to the person skilled in the art in view of the teachings of the present description and as such, will not be further described here.

Note that titles or subtitles may be used throughout the present disclosure for the convenience of the reader, but in no way should these limit the scope of the invention. Moreover, certain theories may be proposed and disclosed herein; however, in no way should they, whether they are right or wrong, limit the scope of the invention so long as the invention is practiced according to the present disclosure without regard for any particular theory or scheme of action.

Any and all references cited throughout the specification are hereby incorporated by reference in their entirety for all purposes.

It will be understood by those of skill in the art that throughout the present specification, the term “a” used before a term encompasses embodiments containing one or more of what the term refers to. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions, will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

As used herein, the term “media”, which also can be referred as the term “fluid”, refers to a material that has a mass and that can be delivered to and travel through an ear canal. For example, the media can be delivered to an ear canal to move ear wax, deliver a drug, or have a cleaning effect within the ear canal. The media can be in various forms, such as, a liquid, a gas, a solid, a colloid, or a gel. The media can be a combination of different substances, such as a mixture of different liquids, gases, or solids, or any combination thereof. For example, the media can be a liquid solution containing a solute such as a detergent or a drug. The media can exhibit a property, such as different viscosities, different pH levels, different salinity and different concentrations. In some embodiments, a variety of media can be used. In some embodiments, it may be water, oil, a detergent, hydroperoxide, alcohols, or a solution containing any combination thereof. In certain embodiments, an individual can purchase a media or a solution over-the-counter. In certain embodiments, an individual may use water such as room temperature water, DI water, or tap water. In some embodiments, an individual may use water or a solution that is sterilized or sanitized. In some embodiments, an individual may use a solution that contains pH buffer or salt to be a saline solution.

### DETAILED DESCRIPTION OF ASPECTS AND EMBODIMENTS OF THE DISCLOSURE

The present disclosure is related to devices for an ear care. In some embodiments, the device is configured to deliver media or fluid to an ear such as an ear canal. Ear wax cleaning devices are tools that are designed to remove earwax from the ear canal. In some embodiments, different types of ear wax cleaning devices may include earwax loops, carwax candles, ear irrigation kits, devices with cameras or sensors, etc., or any combination thereof. Ear irrigation kits use a bulb syringe or a special irrigating device to flush earwax out of the ear canal with irrigating fluid or media.

In some embodiments, a device may include a supply channel to supply media to the ear canal and a drain channel to drain from the ear canal the media supplied to the ear canal.

In some embodiments, a device may include a body having an insertion portion to be inserted into an ear canal. The insertion portion include a supply channel to supply media to the ear canal and a drain channel to drain from the ear canal the media supplied to the car canal. In some embodiments, the supply channel can be located closer to an ear canal than the drain channel when the insertion portion is inserted into the ear canal. In some embodiments, the insertion portion to be inserted into an ear canal includes an outer wall to face the ear canal when the insertion portion is inserted into the ear canal, the drain channel and the supply channel located closer to the outer wall than the drain channel.

In some embodiments, the device may comprise a body having an insertion portion to be inserted into an ear canal, wherein the insertion portion may include an outer wall to face the ear canal when the insertion portion is inserted into the ear canal, a supply channel to supply media to the ear canal when the insertion portion is inserted into the ear canal, and a drain channel to drain the media supplied to the ear canal, wherein the supply channel may be disposed to be closer to the outer wall than the drain channel.

In some embodiments, the device may comprise a body having an insertion portion to be inserted into an ear canal, wherein the insertion portion may include an outer wall to face the ear canal when the insertion portion is inserted into the ear canal, an inner wall forming a space in between the outer wall and the inner wall, a supply channel formed in the space in between the outer wall and the inner wall, the supply channel to supply media to the ear canal when the insertion portion is inserted into the ear canal, and a drain channel formed on the inner side of the insertion portion, the drain portion to drain the media supplied to the ear canal.

In some embodiments, the device may comprise a body having an insertion portion to be inserted into an ear canal, wherein the insertion portion may include an outer wall to face the ear canal when the insertion portion is inserted into the ear canal, an inner wall facing an inner side of the insertion portion, a supply channel formed in between the outer wall and the inner wall, the supply channel to supply media to the ear canal when the insertion portion is inserted into the ear canal, and a drain channel formed on the inner side of the insertion portion, the drain channel to drain the media supplied to the ear canal. In some embodiments, the supply channel may comprise a plurality of supply channels, and wherein the plurality of supply channels may be disposed to be around the circumference of the drain channel.

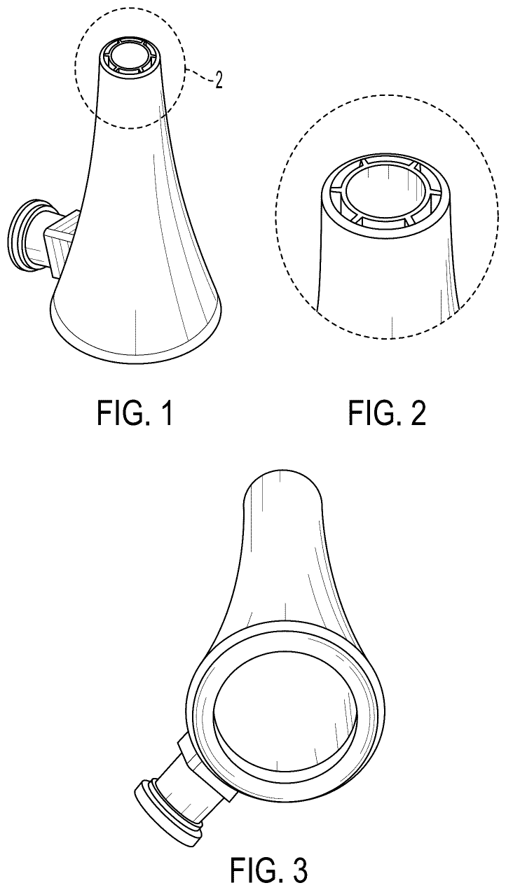

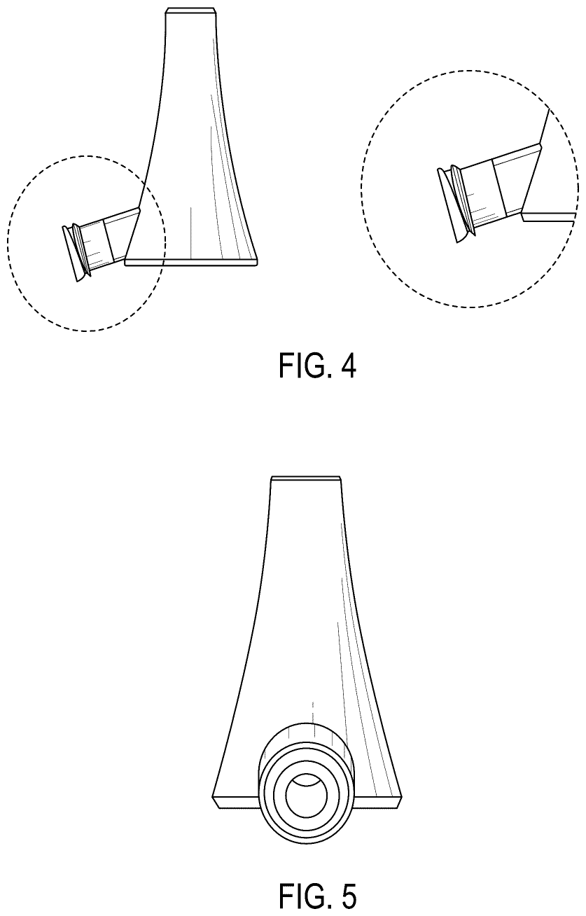

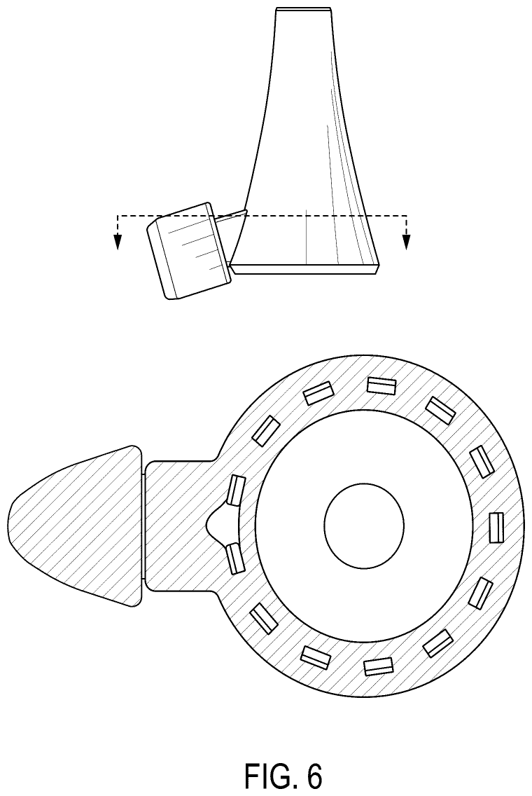

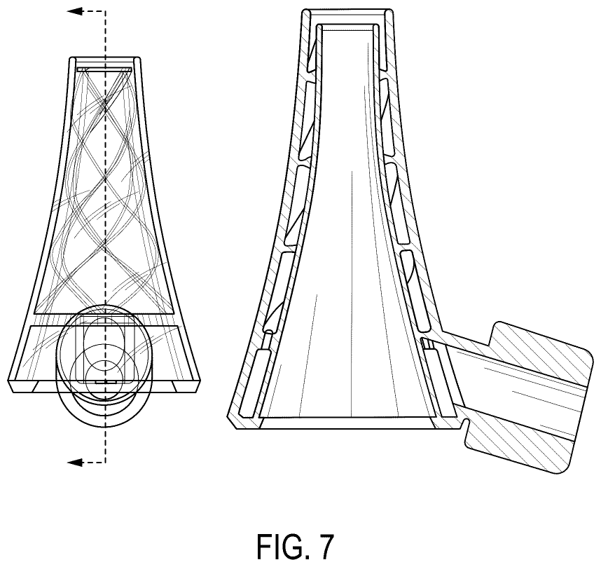

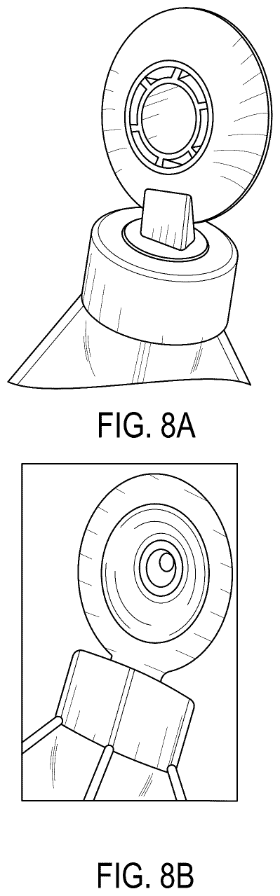

FIGS. 1-5 are schematics of different aspects of a device body having an insertion portion to be inserted into an ear canal according to certain embodiments of this disclosure. FIGS. 6-7 and 8A-8B depict cross-sections of the device body having the insertion portion according to certain embodiments of this disclosure. Referring to FIGS. 1-7 and 8A-8B, the device may include a dual walled cannulated speculum as the device body may include an insertion portion. In certain embodiments, the supply channel may include a portion to direct a flow of the media from the supply channel to generate shear stress on a surface of the ear canal. In certain embodiments, the supply channel may include a portion to direct a flow of the media from the supply channel to be angled toward a surface of the ear canal. Importantly, and in certain embodiments, the supply channel may include centrifugal canals to direct a flow of the media from the supply channel to be in a swirling motion. This is advantageous for the improved operability of the device.

In certain embodiments, the device body comprises a double-walled cannulated irrigating ear speculum; the internal canals can facilitate circular/centrifugal water flow resembling a spiral whirlpool. For example, this cannula, coupled with a generic bulb to serve as the vessel to hold and deliver pressurized water flow, can deliver a powerful stream of swirling water against the walls of the ear canal, to be pushed under the cerumen from the sides and flush it out in a more comfortable manner that prevents a strong stream of uncomfortable pressure directly to the center of the eardrum.

In some embodiments, the dual walled cannulated speculum includes centrifugal canals to deliver the water in a swirling motion, thus, and importantly, the pressurized water never directly hits the eardrum. When water or anything for that matter hits the ear drum, it hurts; this makes it difficult to use other methods and devices currently on the market, particularly for children. In certain embodiments, this method swirls the water against the edges of the ear canal, getting behind the ear wax and pushing it out. This can be accomplished, for example, with a double walled cannulated irrigating ear speculum, with internal canals that will facilitate circular/centrifugal water flow resembling a spiral whirlpool. In some embodiments, the dual walled cannulated speculum may further include an open center as the drain channel. When liquid is squirted into the ear, excessive pressure can build up and hurt or damage the ear and there is nowhere for the water to escape (unlike the nose and sinuses, where it escapes through the mouth or other nostril), this speculum allows the water and cerumen to flow out the open center as the drain channel, accommodating the pressurized flow into the ear (since it can flow out).

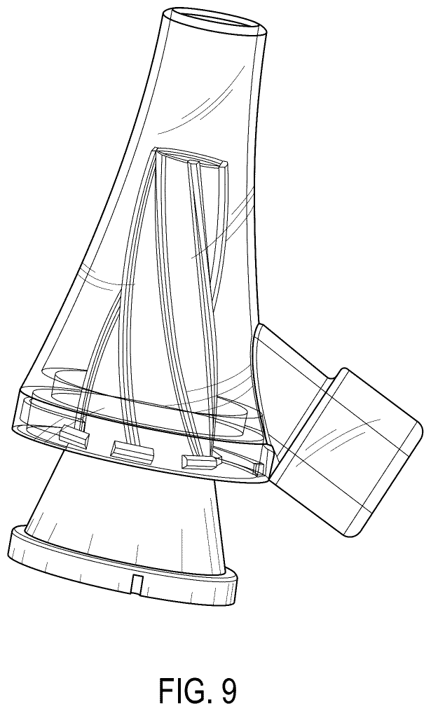

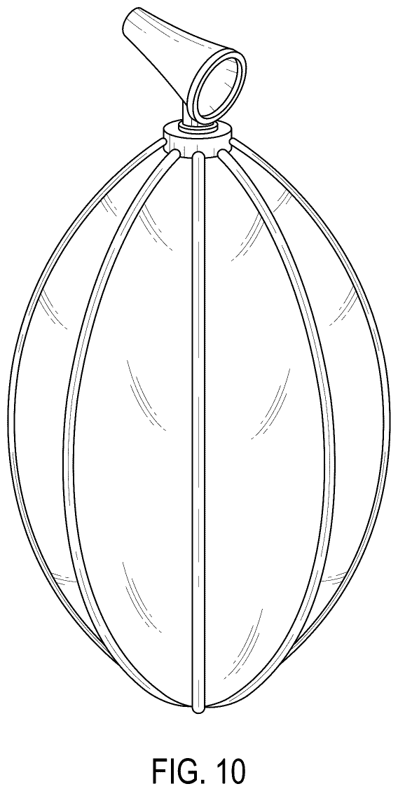

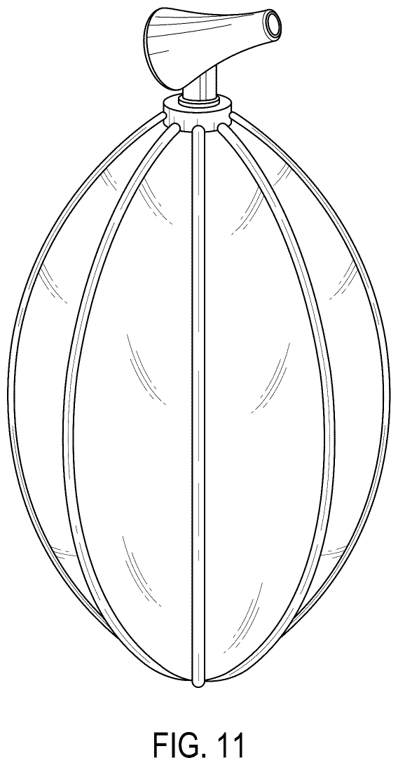

In some embodiments, the device body can be constructed by joining, combining or coupling a plurality of parts, sub-parts, components, or sub-components. In some embodiments, a first component including an outer wall and a second component including an inner wall can be coupled together to form the device body. In some embodiments, the first component or the second components may have a structure to couple and lock the first and second components. In some embodiments, the first component or the second component may include an additional structure, such as portion to form a channel or a drain. For example, FIG. 9 illustrates an example of a device body constructed by coupling an inner component and an outer component of the device body. FIG. 9 indicates an alternate manufacturing method for the speculum in which two parts—an inner core and an outer sheath—are fabricated and then attached in order to create the walls of the speculum. In some embodiments, this may not change the overall design or function of the device but allows for multiple manufacturing processes.

In certain embodiments, the device may further comprise a media container coupled to the body, the media container to contain the media to be supplied to the supply channel.

In certain embodiments, the media container may be squeezable to supply the media to the supply channel. In some embodiments, the media container may be a squeezable bulb to be squeezable to supply the media to the supply channel.

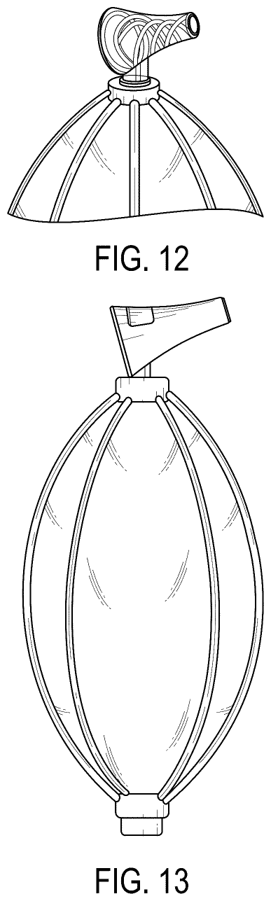

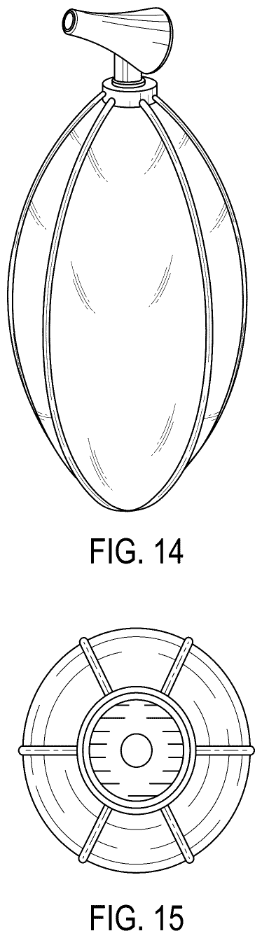

For example, FIGS. 10-12 illustrate different aspects of the device body coupled to a media container, wherein the media container is squeezable to apply pressure to supply the media according to certain embodiments of the disclosure. FIGS. 13-15 are illustrations of an example of this device, where the device body is coupled to the media container. Referring to FIGS. 10-15, in some embodiments, a squeeze bulb pump, for example, made with silicone rubber, can be used as the media container for irrigating the speculum as the device body. In some embodiments, the irrigating speculum uses a soft silicone rubber squeeze bulb to disperse media such as a liquid. The speculum fits easily but snuggly in the bulb opening after it has been filled with the media such as liquid. The speculum is held to the ear as the bulb is squeezed. Excess media then flows back out of the ear through the center hole in the speculum.

In certain embodiments, the bulb can be filled with the media, such as room temperature water. A user of the device can insert the insertion portion of the device body (the speculum) into an ear canal and squeeze bulb firmly to deliver pressurized water into the car canal. For example, a user can lean over a sink, and squeeze the bulb firmly to deliver pressurized water flow into their ear to loosen up the cerumen and push it out.

Currently, when an ENT performs a microscopic cerumen debridement using the suction technique, four things have to be held in their hands at once: (1) a speculum (which they usually let go after they set it in the ear canal, but sometimes need to continue to hold it to move around within the ear canal); (2) a microscope, to guide the speculum to where it needs to point to (also typically let go once it is pointed in the right direction, but again sometimes this needs to be held on to move around to optimize the pointing direction); (3) a saline delivery device, to deliver saline into the ear canal to soften and dilute the wax to allow for its removal through a suction tube (this must be held on to at all times); and (4) a suction tube used to aspirate and remove the saline and wax. The requirement to hold all of these objects at once—at least for a portion of the procedure—is cumbersome and difficult.

Here, it has been demonstrated that this device, allows for the speculum and saline delivery device to be combined into one object that can be held in one hand. I

In some embodiments, the saline flow control system may use a peristaltic pump 600 to disperse liquid at variable speed through the use of a foot pedal 800. The foot pedal 800 contains a potentiometer 803 that is connected with a three-wire cable to a microcontroller 703. The microcontroller 703 outputs a pulse-width modulation signal that controls the speed of the pump 600.

In some embodiments, the device may further comprise a pump 600 to apply a pressure to the media container to supply the media to the supply channel. In some embodiments, the pump 600 may be an electric pump. In some embodiments, the pump 600 may include a pedal 800 to be pressable to generate the pressure. In some embodiments, the device may further comprise a coupler to couple the device to a secondary device.

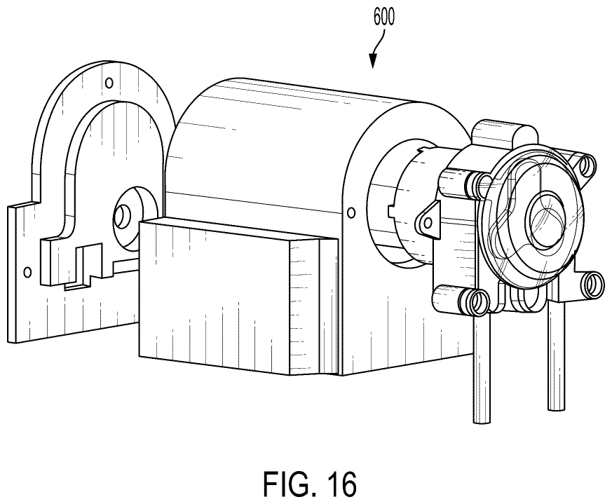

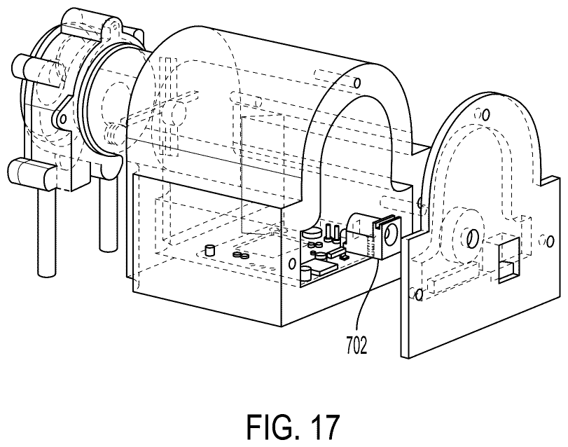

In certain embodiments, an electric or manually operated pump can suck room temp water into the bulb disclosed above or to the device body such as a speculum, such as a continuous water flow by affixing tubing to the device body, with the other end of the tube providing a water source. For example, FIGS. 16-17 illustrate a pump that is operated by electric power in some embodiments of the disclosure.

By having the foot pedal 800 or other means to operate the pump 600 without using a hand, the physician can then deliver the saline through the speculum, alleviating their necd to hold all four things at once.

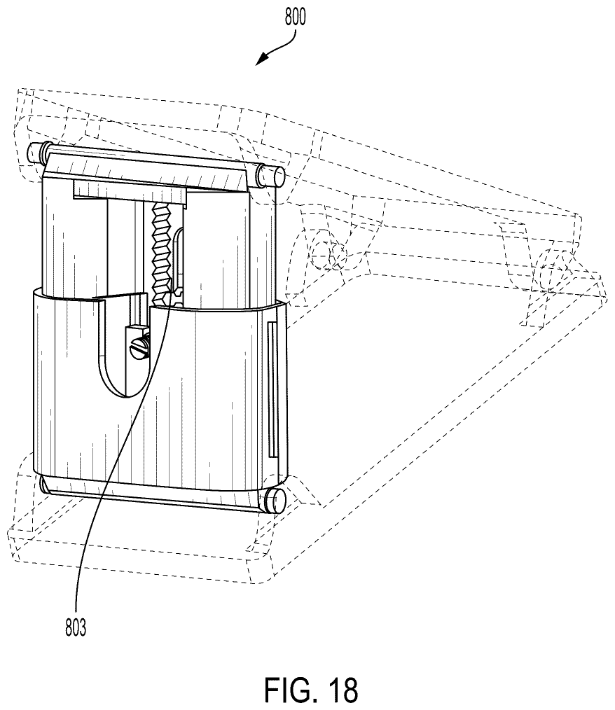

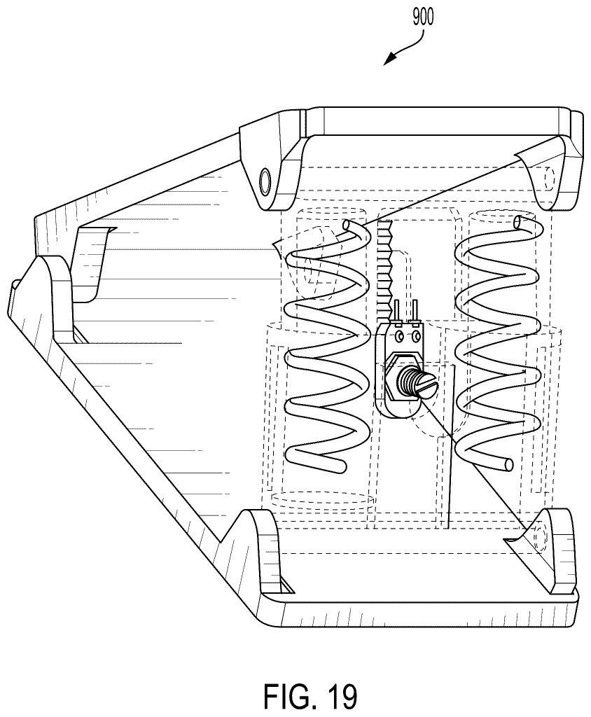

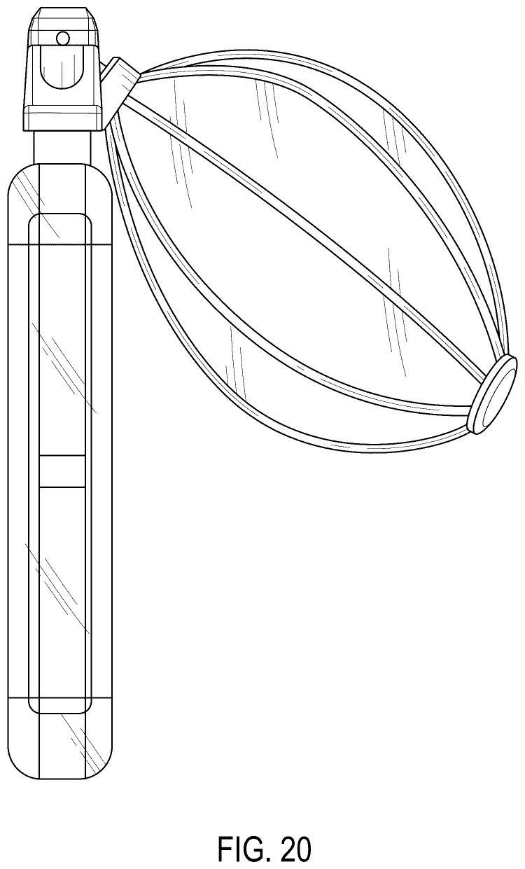

In certain embodiments, the device body such as a speculum can attach to a saline bag cord, whereby the saline would be delivered by applying pressure to a foot pedal. For example, FIGS. 18-19 illustrate a foot pedal to operate the pump 600 according to certain embodiments of the disclosure.

In some embodiments, the device may further comprise a coupler to couple the device to a secondary device. In some embodiments, the secondary device may be an otoscope. FIG. 20 illustrates the device having the device body and the media container are coupled to an otoscope according to certain embodiments of the disclosure.

For example, the device body, such as a speculum, could attach to an otoscope. This version of the speculum has a slanted notch that allows it to be screwed on to an otoscope. The squeeze bulb or other related device can then be attached directly to the speculum.

In some embodiments, when a general practice (GP) physician looks in an individual's ear, they use an otoscope. The otoscope must be equipped with a speculum. A standard disposable speculum may open up the ear canal and direct light into the ear canal allowing for visualization of the ear drum. A standard disposable speculum may guide the light into the direction it is pointed. The standard disposable speculum may be sanitary, so that the otoscope can be used over and over again, by just changing the speculum. It is very common that there is cerumen blocking the ear drum, so general practice physicians are unable to see it. Most of the time, when this happens, the GPs are forced to send the patient to an ENT specialist, to clean out the ear before the ear drum can be seen, or they may resort to using methods that are painful or ineffective.

Accordingly, in certain embodiments, the device can be in a form of speculum that can supply media. The device as described can be notched, to fit the otoscope. The device will be able to replace the currently used, basic speculum. The device will attach to the otoscope to replace the existing basic speculum and satisfy the three purposes listed above. In addition, it will provide the GP with the ability to flush out obstructive wax. To do so, they will simply remove the otoscope, deliver the pressurized water as described herein, and after the water drains out, re-insert the otoscope to have a look at the ear drum.

What is claimed is:

1. A device comprising:
a body having an insertion portion to be inserted into an ear canal,
wherein the insertion portion includes:
an outer wall to face the ear canal when the insertion portion is inserted into the ear canal,
an inner wall facing an inner side of the insertion portion,
a supply channel formed between the outer and inner walls, the supply channel configured to supply media to the ear canal when the insertion portion is inserted into the ear canal, and
a drain channel formed on the inner side of the insertion portion, the drain channel configured to drain the media supplied to the ear canal.

2. The device of claim 1, wherein the supply channel includes a portion to direct a flow of the media from the supply channel to generate shear stress on a surface of the ear canal.

3. The device of claim 1, wherein the supply channel includes a portion to direct a flow of the media from the supply channel to be angled toward a surface of the ear canal.

4. The device of claim 1, wherein the supply channel includes centrifugal canals to direct a flow of the media from the supply channel.

5. The device of claim 4, wherein the flow of the media from the supply channel is a resultant swirling motion.

6. The device of claim 1, wherein the supply channel comprises a plurality of supply channels, and wherein the plurality of supply channels is disposed around the circumference of the drain channel.

7. The device of claim 1, further comprising a media container coupled to the body to supply media to the supply channel.

8. The device of claim 7, wherein the media container is squeezable.

9. The device of claim 7, wherein the media contains is a squeezable bulb.

10. The device of claim 7, further comprising a pump to apply a pressure to the media container to supply media to the supply channel.

11. The device of claim 10, wherein the pump is an electric pump.

12. The device of claim 10, wherein the pump includes a pressable pedal.

13. The device of claim 1, further comprising a coupler to couple the device to a secondary device.

14. The device of claim 13, wherein the secondary device is an otoscope.

15. A device comprising:
a body having an insertion portion to be inserted into an ear canal,
wherein the insertion portion includes:
an outer wall to face the ear canal when the insertion portion is inserted into the ear canal,
an inner wall forming a space between the outer and inner walls,
a supply channel formed in the space in between the outer wall and the inner wall, the supply channel to supply media to the ear canal when the insertion portion is inserted into the ear canal, and
a drain channel formed on the inner side of the insertion portion, the drain channel configured to drain the media supplied to the ear canal.

16. The device of claim 15, wherein the supply channel includes a portion to direct a flow of the media from the supply channel to generate shear stress on a surface of the ear canal.

17. The device of claim 15, wherein the supply channel includes a portion to direct a flow of the media from the supply channel to be angled toward a surface of the ear canal.

18. The device of claim 15, wherein the supply channel includes centrifugal canals to direct a flow of the media from the supply channel.

19. The device of claim 18, wherein the flow of the media from the supply channel is a resultant swirling motion.

20. The device of claim 15, wherein the supply channel comprises a plurality of supply channels, and
wherein the plurality of supply channels is disposed to be around the circumference of the drain channel.

21. A device comprising:
a body having an insertion portion to be inserted into an ear canal,
wherein the insertion portion includes:
an outer wall to face the ear canal when the insertion portion is inserted into the ear canal,
a supply channel to supply media to the ear canal when the insertion portion is inserted into the ear canal, and
a drain channel to drain the media supplied to the ear canal, wherein the supply channel is disposed to be closer to the outer wall than the drain channel.

22. The device of claim 21, wherein the supply channel includes a portion to direct a flow of the media from the supply channel to generate shear stress on a surface of the ear canal.

23. The device of claim 21, wherein the supply channel includes a portion to direct a flow of the media from the supply channel to be angled toward a surface of the ear canal.

24. The device of claim 21, wherein the supply channel includes centrifugal canals to direct a flow of the media from the supply channel.

25. The device of claim 24, wherein the flow of the media from the supply channel is a resultant swirling motion.
